(19) United States
(12) Patent Application Publication
(10) Pub. No.: US 2025/0194903 A1
(43) Pub. Date: Jun. 19, 2025

(54) TORQUE DEVICE

(71) Applicant: The Regents of the University of California, Oakland, CA (US)

(72) Inventors: Shanglei Liu, San Diego, CA (US); Mary Lee Krinsky, San Diego, CA (US); Nathan Delson, San Diego, CA (US); Andrew Zhang, Basking Ridge, NJ (US); Hyeongmin Kim, San Diego, CA (US)

(21) Appl. No.: 18/866,955

(22) PCT Filed: May 19, 2023
(86) PCT No.: PCT/US23/22927

§ 371 Date: Nov. 18, 2024

Related U.S. Application Data
(60) Provisional application No. 63/344,476, filed on May 20, 2022.

(51) Int. Cl. A61B 1/00

(52) U.S. Cl. CPC A61B 1/00131; A61B 1/00147

(57) ABSTRACT

A device includes a body extending along a body axis from a first body end to a second body end. The body includes a first component defining a first outer surface and first groove extending along the body axis. The body further includes a second component coupled to the first component such that the first component pivotable relative the second component between an open configuration and a closed configuration, the second component defining a second outer surface and a second groove extending along the body axis. The first groove and the second groove cooperating to define a channel extending within the body proximate the first body end along the body axis when the first component and the second component are in the closed configuration, the channel having a channel height and a channel width.

### CROSS-REFERENCE TO RELATED APPLICATION

This application claims the benefit of and priority to U.S. Provisional Patent Application No. 63/344,476, filed May 20, 2022, the contents of which is incorporated herein by reference in its entirety.

### BACKGROUND

Medical tubes can be difficult to grip, torque, and/or manipulate. During certain procedures, such as endoscopic procedures, a lubricant may be applied to the outside of the medical tube, which may increase difficulties with grip, torque, and/or manipulation of the medical tube

### SUMMARY

A set of embodiments relate to a device including a body extending along a body axis from a first body end to a second body end. The body includes a first component defining a first outer surface and first groove extending along the body axis. The body further includes a second component coupled to the first component such that the first component pivotable relative the second component between an open configuration and a closed configuration, the second component defining a second outer surface and a second groove extending along the body axis. The first groove and the second groove cooperating to define a channel extending within the body proximate the first body end along the body axis when the first component and the second component are in the closed configuration, the channel having a channel height and a channel width.

Another set of embodiments relate to a body extending along a body axis from a first body end to a second body end, the body being manipulable between an open configuration and a closed configuration. The body defines an outer surface and a channel extending within the body proximate the first body end and along the body axis. The body further defines a gap extending from the outer surface into the channel, the gap being larger when the body is in the open configuration than the closed configuration the channel defining a polygon cross-section.

### BRIEF DESCRIPTION OF THE DRAWING

FIG. 1 illustrates a perspective view of a torque device, according to an example embodiment.

FIG. 2 illustrates another perspective view showing a portion of the torque device of FIG. 1.

FIG. 3 illustrates a side view of a component of the torque device portion of FIG. 2.

FIG. 4 illustrates a perspective view of another torque device, according to an example embodiment.

FIG. 5 illustrates another perspective view of the torque device of FIG. 4.

FIG. 6A illustrates a front view of the torque device of FIG. 4

FIG. 6B illustrates a front view of the torque device of FIG. 4 along with a force clamp.

FIG. 7 illustrates a front view of a component of a torque device, according to an example embodiment.

FIG. 8 illustrates a front view of a component of another torque device, according to an example embodiment.

FIG. 9 illustrates a perspective view of another torque device, according to an example embodiment.

FIG. 10 illustrates a perspective view of a component of another torque device, according to an example embodiment.

FIG. 11 illustrates a perspective view of a component of another torque device, according to an example embodiment.

FIG. 12 illustrates a perspective view of another torque device, according to an example embodiment.

FIG. 13 illustrates a front view of the torque device of FIG. 12.

### DETAILED DESCRIPTION

In the following detailed description, reference is made to the accompanying drawings, which form part of the present disclosure. The embodiments described in the drawings and description are intended to be example and not limiting. As used herein, the term “example” means “serving as an example or illustration” and should not necessarily be construed as preferred or advantageous over other embodiments. Other embodiments may be utilized and modifications may be made without departing from the scope of the subject matter presented herein. Aspects of the disclosure, as described and illustrated herein, can be arranged, combined, and designed in a variety of different configurations, all of which are explicitly contemplated and form part of this disclosure.

Medical tubes can be difficult to grip, torque, and/or manipulate. Further, repeated gripping, torqueing, and/or manipulation of the medical tube may result in undesired health implications for the user of the medical tube. For example, physicians that regularly perform endoscopy procedures may suffer from relatively high rates of work-related injury, with 37%-89% reporting a musculoskeletal injury, and 21% seeking medical care. Work-related injuries can shorten the career and reduce the number of endoscopy procedures the physicians are able to perform.

A colonoscopy can be one of the most common and physically rigorous endoscopic procedures. During this procedure, physicians apply high forces to grip, torque, and manipulate a thin and often lubricated endoscope. This may be a source of fatigue and may lead to repetitive stress injury.

Referring to the figures generally, one aspect of the disclosure is directed towards a torque device (e.g., a gripping device, a hand gripping device, etc.). The torque device includes a channel configured to receive a medical tube (e.g., a flexible tube of an endoscope, an insertion tube of an endoscope, etc.) and an outer surface (e.g., a grip surface) the user (e.g., physician) can squeeze to better grip the medical tube for insertion, torqueing, and/or manipulation of the medical tube.

The torque devices disclosed herein may allow for reduced stress to the user of the endoscope. The torque devices may increases the perceived diameter of the medical tube so that care taker performing medical procedure (e.g., endoscopy) are able to apply torque to the scope easier, thereby decreasing the stresses experienced in their hand and arms. The torque devices may increase the diameter of the portion of the medical tube usually held by the operator to give the user mechanical advantage when gripping, torqueing, and/or manipulating the medical tube. The torque device may include finger holds, may be squeezable, may have an angled grip surface, and/or the grip surface may have a relatively high coefficient of friction for improved grip. The grip surface may additionally include channels for receiving lubricants and debris.

The torque device may include a flange at the end to accommodate bends or curvature in the medical tube (e.g., endoscope curvature) and/or prevent slipping out of the device. The device can be attached to the scope, made part of a glove, and/or be a standalone device. The device can be disposable or reusable.

The device may be short enough so that the last two fingers of the hand using it can follow the natural contour of the hand and rest on the scope rather than be fully on the gripping device. The device can use as either a disposable or reusable part sold to endoscopy companies, hospital that perform endoscopes, or doctors that perform endoscopies.

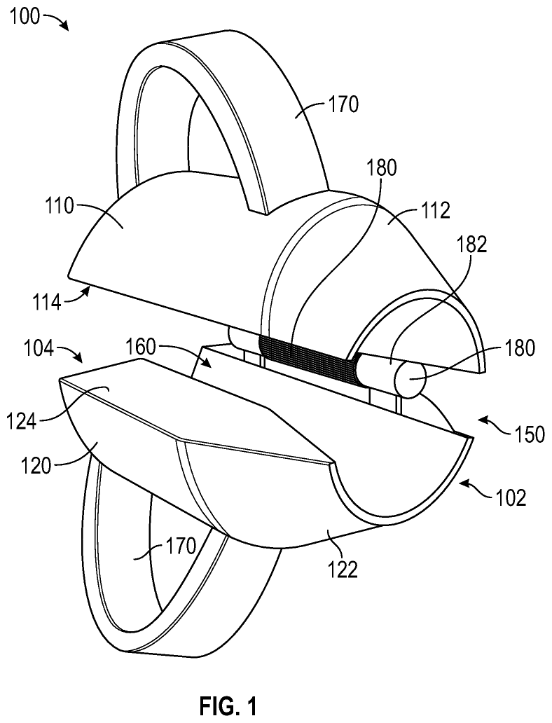

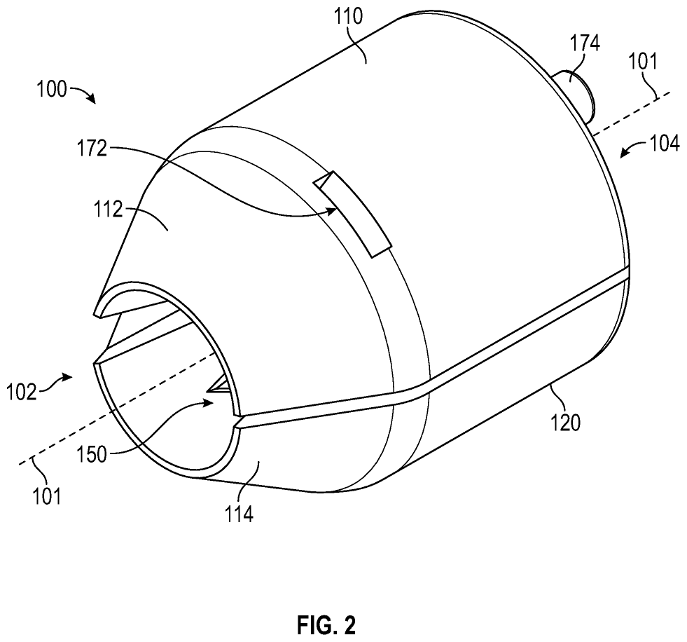

Referring now to FIGS. 1 and 2, perspective views of a torque device 100 are shown. The torque device 100 includes a body that extends along a body axis 101 between a first body end 104 and a second body end 102. The body includes a first component 110 and a second component 120 coupled to the first component 110. The body can be manipulated between an open configuration (e.g., shown in FIG. 1) and a closed configuration (e.g., shown in FIG. 2). When the torque device 100 the open configuration, the distance between an interior surface 114 of the first component 110 and an interior surface of the second component 124 (e.g., a gap distance) is larger than when the torque device 100 is in the closed configuration.

The torque device 100 includes a channel 160 configured to receive the medical tube such that the torque device 100 can be repositioned along the medical tube. The channel 160 is defined by a first groove in the first component 110 and a second groove in the second component 120 when the torque device 100 is in the closed configuration. The channel 160 extends into the body of the torque device 100 proximate the first body end 104 along the body axis 101.

The torque device 100 may be manipulated into the open configuration to a medical tube to be inserted into the channel 160 of the torque device 100. The torque device 100 can then manipulated into the closed configuration such that one or more walls of the channel 160 contact the medical tube so that the torque device 100 can be utilized to manipulate the medical tube while the medical tube is positioned within the channel 160.

The first component 110 is coupled to the second component 120 via a pair of hinges 180. The pair of hinges 180 allow the first component 110 to pivot with respect to the second component 120 about the hinges 180. The torque device further includes a biasing structure, shown as a spring 182 proximate the hinges 180. The spring 182 is configured to bias the torque device 100 towards the closed configuration. According to various examples, a user (e.g., a physician) may remove and replace his or her hand from the torque device 100 (e.g., to take a closer look at the camera screen, to grab another tool, etc.). The spring 182 biases the torque device towards the closed configuration so that the channel 160 continues to engage the medical tube even while there is no external clamping force being applied by the user. According to various embodiments, biasing the torque device 100 toward the closed configuration may reduce slippage of the torque device on the medical tube while the user removes his or her hand from the torque device 100.

Referring to FIG. 1, the torque device 100 further includes a first strap 170 (e.g., loop, hook, etc.) coupled to an exterior surface of the first component 110 and a second strap 170 coupled to an exterior surface of the second component 120. As shown in FIG. 2, the first component 110 includes a strap aperture 172 configured to receive a first end of the first strap 170 and a projection 174 configures to couple to a second end of the first strap 170 to couple the first strap 170 to the first component. The first strap 170 and the second strap 170 are configured to receive a hand of the user such that the user can utilize the first strap 170 and the second strap 170 to open and/or close the torque device 100.

The first exterior surface of the first component 110 and the second exterior surface of the second component 120 define a round outer surface (e.g., the gripping surface). The round outer surface defines an outer surface diameter (e.g., a maximum outer diameter). Further, the channel 160 defines a channel height and a channel width configured to accommodate a medical tube. For example, at least one of the channel height or the channel width may be sided to accommodate a desired medical tube having a medical tube diameter. According to various embodiments, the outer surface diameter is at least twice as large as the medical tube diameter the torque device 100 is configured to receive. Thus, the outer surface diameter is at least twice as large as the channel width and/or the channel height. Further, the outer surface diameter may be approximately four times as large as the medical tube diameter the torque device 100 is configured to receive. Thus, the outer surface diameter is at least four times as large as the channel width and/or the channel height.

The first component 110 and the second component 120 collectively define an interior conical portion 150 (e.g., a funnel) proximate the second body end 102. The conical portion 150 and the channel 160 collectively form a body aperture when the torque device 100 is in the closed configuration. The body aperture extends along the body axis 101 from the first body end 104 to the second body end 102 such that the torque device 100 can translate along a medical tube while remaining in the closed configuration.

The conical portion 150 is configured to accommodate a curve in the medical tube. For example, the medical tube may have a smaller outer diameter than at least a portion of the inner diameter of the conical portion 150 such that the medical tube can bend within the conical portion 150. The conical portion 150 extends along the body axis 101 into the body from the second body end 102 to channel 160 when then torque device 100 is in the closed configuration. The inner diameter of the conical portion 150 is greatest proximate the second body end 102 and decreases as the conical portion 150 extends into the body (e.g., the inner diameter of the conical portion 150 is smallest proximate the channel 160).

The first component 110 includes a first tapered exterior surface 112 proximate the second body end 102. Additionally, the second component 120 includes a second tapered exterior surface 122 proximate the second body end 102. The first tapered exterior surface 112 and the second tapered exterior surface 122 collectively define a tapered outer portion proximate the second body end 102.

The tapered outer portion proximate the second body end 102 may allow for more comfortable positioning of the hand on the torque device 100. The tapered outer portion, in combination with the grip area being relatively short (e.g., the distance between the first body end 104 and the second body end 102 being shorter than the width of an average hand) allows for the user to place part of his or her hand on the grip while still leaving part of his or her hand on the medical tube (e.g., endoscope). Thus, the tapered outer portion may enhance the user's grip as well as provide a more tactile sensation from more direct contact between the user's hand and the medical tube (e.g., endoscope).

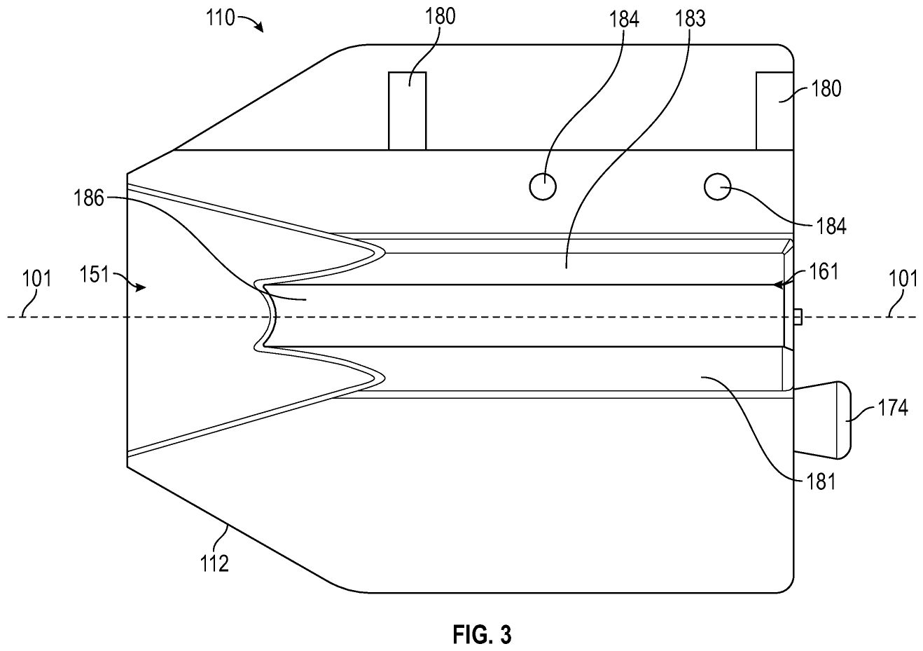

Referring now to FIG. 3, a side view of the first component 110 is shown. The first component 110 includes a first groove 161 extending along the body axis 101 though the first component 110. The first groove 161 includes a conical portion 151 that defines a part of the conical portion 150 of the torque device 100 when the torque device 100 is in the closed configuration. The groove 161 further includes a first sidewall 181, a second sidewall 183, and an end wall 186 extending from the first sidewall 181 to the second sidewall 183. The first sidewall 181, the second sidewall 183, and the end wall 186 collectively define a portion of the channel 160 when the torque device 100 is in the closed configuration.

Further, the first component 110 includes a pair of spring apertures 184 extending into the first component 110. Each of the spring apertures 184 are configured to receive an end of a spring or similar biasing member (e.g., the spring 182). As discussed above, the spring may be configured to bias the torque device 100 towards the closed orientation.

It should be appreciated that the second component 120 may include features similar to the features as the features shown in FIG. 3. For example, the second component 120 may define a second groove having a first sidewall, a second sidewall, and an end wall connecting the first sidewall to the second sidewall. In this example embodiment, the channel 160 defined by the first component 110 and the second component 120 will have a six sided polygon cross section when the torque device 100 in in the closed configuration. According to other embodiments, the channel 160 defines a polygonal cross section having eight sides or less.

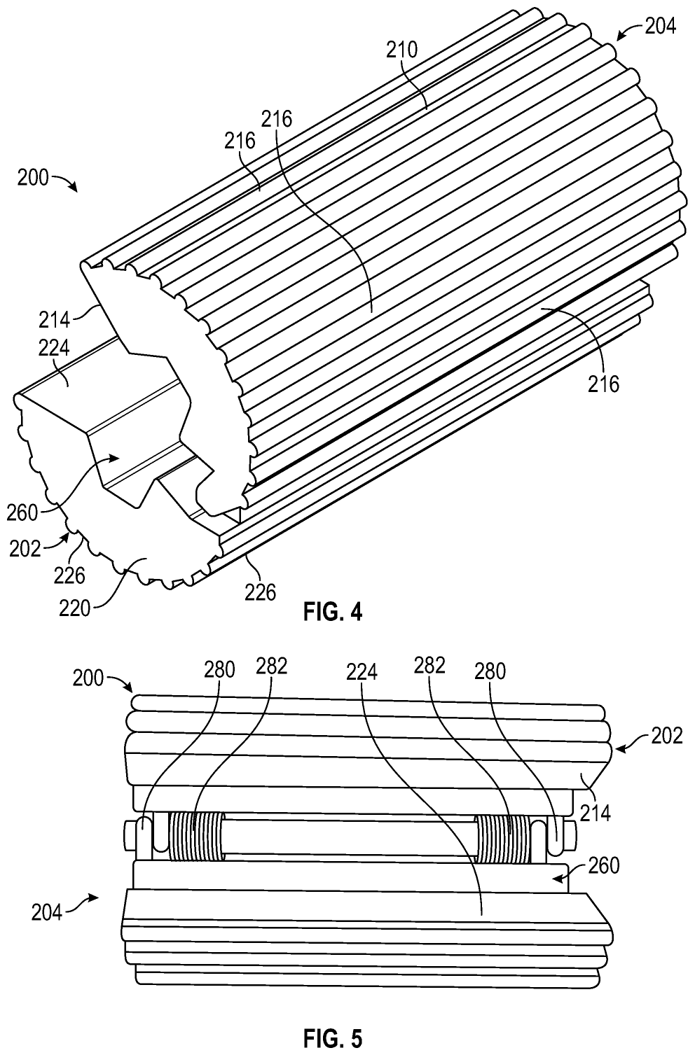

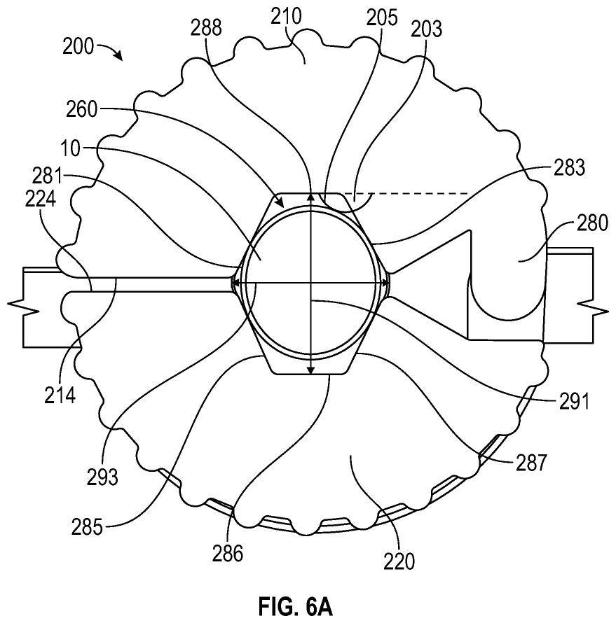

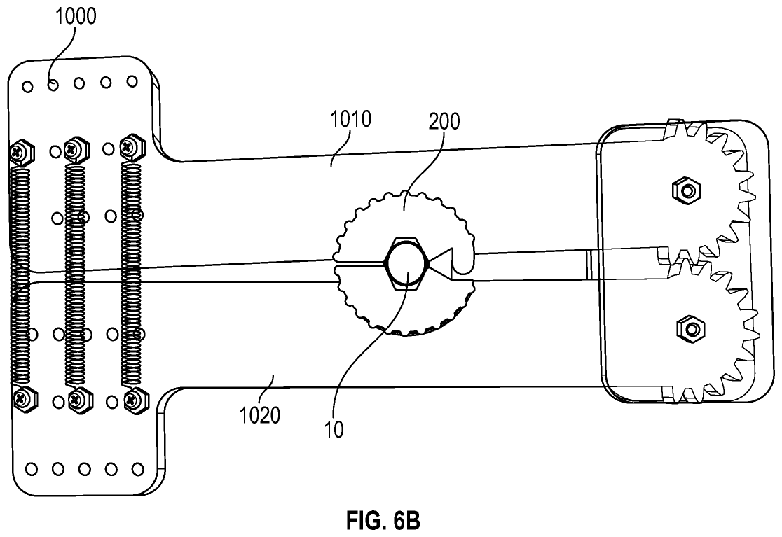

Referring now to FIGS. 4 and 5, perspective views of another torque device 200 are shown, according to an example embodiment. The torque device 200 includes a body that extends along a body axis between a first body end 204 and a second body end 202. The body includes a first component 210 and a second component 220 coupled to the first component 210. The body can be manipulated between an open configuration (e.g., shown in FIGS. 4 and 5) and a closed configuration (e.g., shown in FIG. 6A). When the torque device 200 the open configuration, the distance between an interior surface 214 of the first component 210 and an interior surface of the second component 224 (e.g., a gap distance) is larger than when the torque device 200 is in the closed configuration.

The torque device 200 includes a channel 260 configured to receive the medical tube such that the torque device 200 can be repositioned along the medical tube. The channel 260 is defined by a first groove in the first component 210 and a second groove in the second component 220 when the torque device 200 is in the closed configuration. The channel 260 extends into the body of the torque device 200 proximate the first body end 204 along the body axis.

The torque device 200 may be manipulated into the open configuration to a medical tube to be inserted into the channel 260 of the torque device 200. The torque device 200 can then manipulated into the closed configuration such that one or more walls of the channel 260 contact the medical tube so that the torque device 200 can be utilized to manipulate the medical tube while the medical tube is positioned within the channel 260.

The first component 210 is coupled to the second component 220 via a pair of hinges 280. The pair of hinges 280 allow the first component 210 to pivot with respect to the second component 220 about the hinges 280. The torque device further includes a pair of biasing structures, shown as springs 282, proximate the hinges 280. Each spring 282 is configured to bias the torque device 200 towards the closed configuration. According to various examples, a user (e.g., a physician) may remove and replace his or her hand from the torque device 200 (e.g., to take a closer look at the camera screen, to grab another tool, etc.). The spring 282 biases the torque device towards the closed configuration so that the channel 260 continues to engage the medical tube even while there is no external clamping force being applied by the user. According to various embodiments, biasing the torque device 200 toward the closed configuration may reduce slippage of the torque device on the medical tube while the user removes his or her hand from the torque device 200.

The first component 210 includes a first plurality of ridges 216 (e.g., grooves, channels, indentations, etc.) on an exterior surface of the first component 210. The second component 220 includes a second plurality of ridges 226 on an exterior surface of the second component 220. The first plurality of ridges 216 and the second plurality of ridges 226 may increase a surface roughness of the round outer surface of the torque device 200, which may increase a user's ability to apply a torque without slipping. Additionally, the first plurality of ridges 216 and the second plurality of ridges 226 may receive lubricant or other debris during the procedure, which may further increase a user's ability to apply a torque without slipping.

Referring now to FIG. 6A, a front view of the torque device 200 is shown. The torque device 200 is shown in the closed configuration with a medical tube 10 positioned within the channel 260. The channel 260 is partially defined by a first groove in the first component 210 (e.g., by a first sidewall 281, a second sidewall 283, and an end wall 288 extending from the first sidewall 281 to the second sidewall 283). The channel 260 is partially defined by a second groove in the second component 220 (e.g., by a first sidewall 285, a second sidewall 287, and an end wall 286 extending from the first sidewall 285 to the second sidewall 287). According to various embodiments, the distance between the end wall 288 and the end wall 286 defines a channel height 291. Further, the channel 260 defines a channel width 293 (e.g., the dimension perpendicular to the channel height).

The first end wall 288 and the second sidewall 283 define an interior angle 205. The interior angle 205 is greater than 90 degrees. The interior angle 205 may be less than 120 degrees. The interior angle 205 may less than 110 degrees. The interior angle 205 may be between 110 and 120 degrees. The interior angle 205 may be approximately 100 degrees. The first sidewall 285 and the end wall 288 further define a wedge angle 203. The wedge angle 203 may be less than 90 degrees. The wedge angle 203 may be 50 degrees or more. The wedge angle 203 may be between 90 degrees and 65 degrees. The wedge angle 203 may be between 75 degrees and 85 degrees. The wedge angle 203 may be approximately 80 degrees

It should be appreciated that first sidewall 281 and the end wall 288 may form a second interior angle that is similar to the interior angle 205. Further, the first sidewall 285 and the end wall 286 may define a third interior angle that is similar to the interior angle 205. Furthermore, the second sidewall 287 and the end wall 286 may define a fourth interior angle that is similar to the interior angle 205.

Referring now to FIG. 6B, the torque device 200 is shown positioned within a first arm 1010 and a second arm 1020 of a force clamp 1000. Additionally, a medical tube 10 is positioned within the torque device 200. The torque device 200 was tested using a medical tube 10 (e.g., a rigid endoscope analog) to measure the effective torque being applied to the medical tube 10 by the torque device 200. Torque was applied to the torque device 200 by the force clamp 1000 to simulate a user's grip while the maximum torque that the torque device 200 can generate before slippage occurs and measurements were taken. A spring preload that approximates 45% of maximum typical gripping force was applied to four different torque devices, each having different wedge angles. The results are summarized in Table 1 below, which shows an increase of up to 19% additional torque before slippage occurs.

TABLE 1

| Wedge Angle | Average Torque at | Torque | Percentage Increase |
|---|---|---|---|
| (°) | Slippage (Nm) | Multiplier | from Baseline |
| 0 | 0.930 | 1 | 0% |
| (Baseline) | | | |
| 50 | 0.987 | 1.061 | 6.1% |
| 65 | 1.030 | 1.107 | 10.7% |
| 80 | 1.107 | 1.190 | 19.0% |

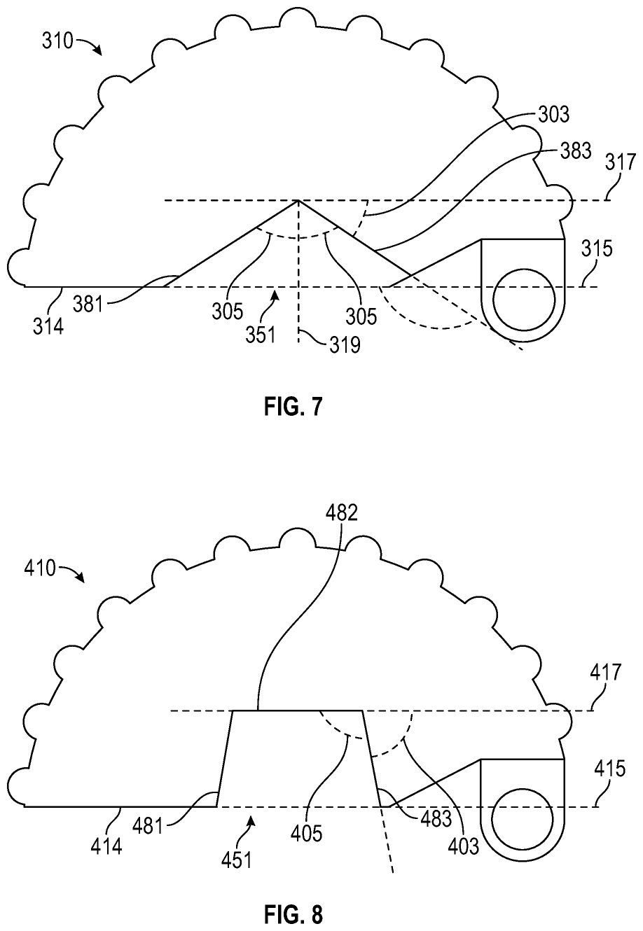

Referring now to FIG. 7, a front view of a component 310 of another torque device is shown, according to an example embodiment. The component 310 may share one or more characteristics with any of the other components (e.g., the first component 110, the second component 120, the first component 210, the second component 220, etc.) described herein.

The component 310 includes a groove 351 extending from a first plane 315 into an interior surface 314 of the component 310. The first plane 315 is parallel to the interior surface 314. The groove 351 is defined by a first sidewall 381 and a second sidewall 383. The first sidewall 381 and the second sidewall 383 intersect within a second plane 317 that is parallel to the interior surface 314. The first plane 315 and the second plane 317 are parallel one another. According to various embodiments, the component 310, when coupled with a complementary component, defines at least a portion of a channel of a torque device. The channel may have a four sided polygon cross section, including the first sidewall 381 and the second sidewall 383.

As shown, the second sidewall 383 defines a wedge angle 303 (e.g., with respect to the second plane 317). The wedge angle 303 may be between 25 degrees and 55 degrees. The wedge angle 303 may be between 30 degrees and 50 degrees. The wedge angle 303 may be between 30 and 35 degrees. The wedge angle 303 may be between 40 and 45 degrees. It should be appreciated that the first sidewall 381 defines a similar wedge angle.

The first sidewall 381 and the second sidewall 383 each define an interior angle 305 (e.g., the angle between the first sidewall 381 or the second sidewall 383 and the plane 319, wherein the plane 319 is perpendicular to the plane 317). Each interior angle 305 may be between 50 degrees and 80 degrees. The interior angle 305 may be between 55 degrees and 75 degrees. The interior angle 305 may be between 50 and 60 degrees. The interior angle 305 may be between 60 and 70 degrees.

Referring now to FIG. 8, a front view of a component 410 of another torque device is shown, according to an example embodiment. The component 410 may share one or more characteristics with any of the other components (e.g., the first component 110, the second component 120, the first component 210, the second component 220, the component 310, etc.) described herein.

The component 410 includes a groove 451 extending from a first plane 415 into an interior surface 414 of the component 410. The first plane 415 is parallel to the interior surface 414. The groove 451 is defined by a first sidewall 481, a second sidewall 483, and an end wall 482 extending between the first sidewall 481 and the second sidewall 483. The end wall 482 extends along a second plane 417 that is parallel to the interior surface 414. The first plane 415 and the second plane 417 are parallel one another.

As shown, the second sidewall 483 defines a wedge angle 403 (e.g., with respect to the second plane 417). The wedge angle 403 may be between 70 degrees and 90 degrees. The wedge angle 403 may be between 75 degrees and 85 degrees. The wedge angle 403 may be approximately 80 degrees. It should be appreciated that the first sidewall 481 defines a similar wedge angle.

The first sidewall 481 and the second sidewall 483 each define an interior angle 405 (e.g., the angle between the first sidewall 381 or the second sidewall 383 and the end wall 482). Each interior angle 405 may be greater than 90 degrees. The interior angles may be between 95 and 110 degrees. The interior angle 405 may be approximately 100 degrees.

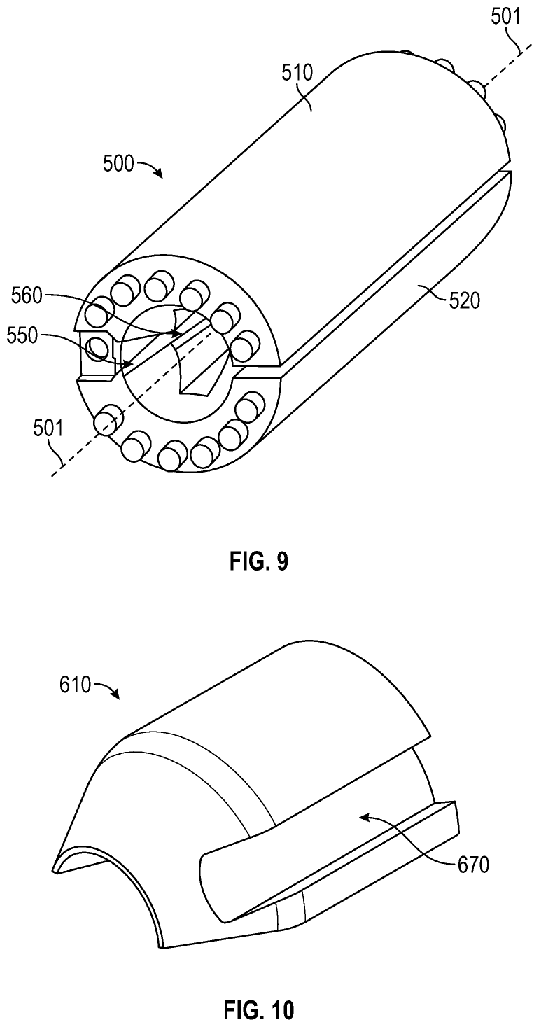

Referring now to FIG. 9, another torque device 500 is shown according to an example embodiment. The torque device 500 extends along a body axis 501 from a first end to a second end. The torque device 500 includes a first component 510 and a second component 520 configured to pivot with respect to one another to transform the torque device 500 between a closed configuration (shown in FIG. 9) and an open configuration.

The torque device 500 includes a body aperture extending through the torque device 500 along the body axis 501. The body aperture is defined by a conical portion 550 and a channel 560. The conical portion 550 and/or the channel 560 may share one or more characteristics with any of the other conical portions or channels described herein. Unlike other torque devices described herein, the torque device 500 has a constant outer diameter when in the closed configuration.

Referring now to FIG. 10, a component 610 of another torque device is shown, according to an example embodiment. The component 610 may share one or more characteristics with any of the other components (e.g., the first component 110, the second component 120, the first component 210, the second component 220, etc.) described herein. The component 610 includes a groove 670 extending into the exterior surface of the component 610 and along a first distance of the component 610. The groove 670 is configured to receive one or more finger tips of a user of the component 610. The user may utilize the groove 670 to open and close the torque device that the component 610 is included in (e.g., instead of utilizing the straps 170 shown in FIGS. 1 and 2).

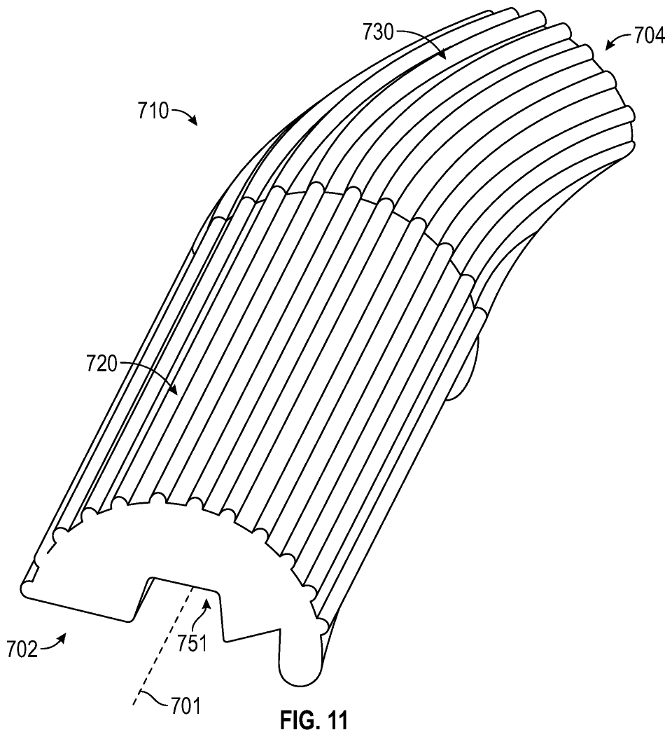

Referring now to FIG. 11, a component 710 of still another torque device is shown, according to an example embodiment. The component 710 may share one or more characteristics with any of the other components (e.g., the first component 110, the second component 120, the first component 210, the second component 220, etc.) described herein. As shown, the component extends from a first body end 704 to a second body end 702. The component 710 includes a straight portion 720 proximate the second body end 702 that extends along a body axis 701. The component 710 further includes a curved portion 730 proximate the first body end 740. The curved portion extends from the straight portion 720 at a curvature with respect to the body axis 701.

According to various embodiments, the curved portion 730 allow for a “jamming” motion of the medical tube inside the component 710 and provides more of a lever arm on the medical tube by forcing a bend in the medical tube, which may increase the amount of force that can be applied to the medical tube without slipping within a groove 751 of the component 710.

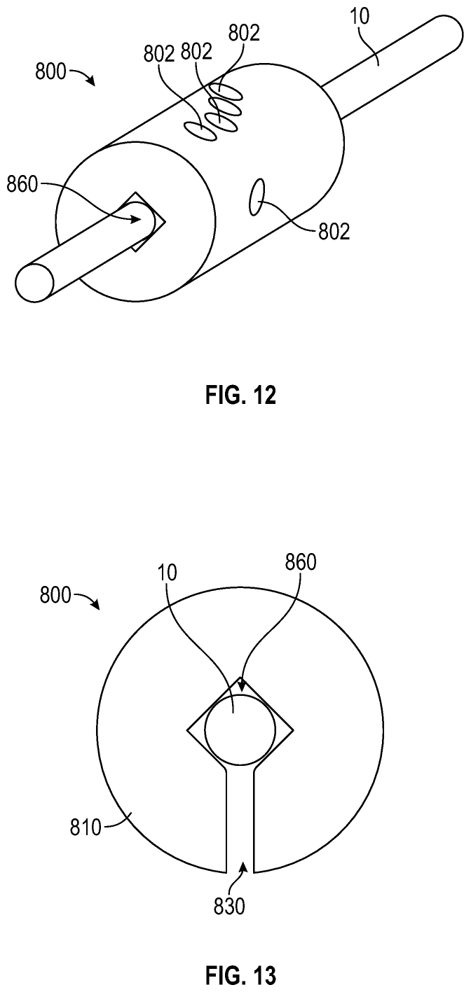

Referring now to FIGS. 12 and 13, another torque device 800 is shown, according to an example embodiment. The torque device 800 may share one or more characteristics with any of the other torque devices described here (e.g., the torque device 100, the torque device 200, etc.). The torque device 800 includes a single integrally formed body 810 that defines a gap 830 and a channel 860. The torque device 800 is configured to transform between an open configuration and a closed configuration (e.g., the gap 830 expands as the torque device 800 transforms from the closed configuration to the open configuration).

The torque device 800 includes a plurality of finger holds 802 configured to receive a user's finger tips to facilitate the user manipulating the torque device 800 between the open configuration and the closed configuration.

The torque device 800 defines the channel 860 configured to receive a medical tube 10. The torque device 800 defines the gap 830 configured allow the medical tube 10 to pass from outside the torque device and into the channel 860. The gap 830 is larger when the torque device 800 is in the open configuration than when the torque device 800 is in the closed configuration.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

The disclosures illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that the materials, methods, and examples provided here are representative of preferred embodiments, are exemplary, and are not intended as limitations on the scope of the disclosure.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

Unless otherwise defined, all technical and scientific terms used herein have the same meaning as commonly understood by one of ordinary skill in the art to which this disclosure belongs.

All numerical designations, e.g., angles and dimensions, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading the present disclosure and so forth.

As used herein, the term “about,” when referring to a measurable value such as an amount or concentration and the like, is meant to encompass variations of 20%, 10%, 5%, 1%, 0.5%, or even 0.1% of the specified amount.

The following examples are intended to illustrate but not limit the disclosure.

1. A device, comprising:
a body extending along a body axis from a first body end to a second body end, the body comprising:
a first component defining a first outer surface and first groove extending along the body axis; and
a second component coupled to the first component such that the first component pivotable relative the second component between an open configuration and a closed configuration, the second component defining a second outer surface and a second groove extending along the body axis, wherein:
the first groove and the second groove cooperating to define a channel extending within the body proximate the first body end along the body axis when the first component and the second component are in the closed configuration, the channel having a channel height and a channel width.

2. The device of claim 1, wherein the first outer surface and the second outer surface define an outer body surface having a body diameter when the first component and the second component are in the closed configuration, the body diameter being at least twice as large as the channel height and the body diameter being at least twice as large as the channel width.

3. The device of claim 1, wherein the body diameter is at least four times as large as at least one of the channel height and the channel width.

4. (canceled)

5. The device of claim 1, wherein the first groove includes a first sidewall, a second sidewall, and an end wall connecting the first sidewall and the second sidewall.

6. (canceled)

7. (canceled)

8. (canceled)

9. (canceled)

10. (canceled)

11. The device of claim 1, wherein the first component is coupled to the second component via a hinge.

12. (canceled)

13. The device of claim 1, wherein the first component defines a first plurality of ridges extending from the first outer surface and the second component defines a second plurality of ridges extending from the second outer surface.

14. The device of claim 1, wherein the first groove and the second groove further define a conical portion proximate the second body end when the first component and the second component are in the closed configuration, the conical portion being in fluid communication with the channel.

15. The device of claim 14, wherein the first component defines a tapered outer portion proximate the second body end.

16. The device of claim 1, wherein the first component and the second component are coupled to a biasing structure configured to bias the first component and the second component towards the closed configuration.

17. The device of claim 1, further comprising:
a first loop coupled to the first outer surface of the first component; and
a second loop coupled to the second outer surface of the second component.

18. The device of claim 1, wherein the first component and the second component define a curved body portion proximate the second body end.

19. The device of claim 1, wherein the first component includes a finger groove in the first outer surface, the finger groove extending in a direction parallel to the body axis.

20. A device, comprising:
a body extending along a body axis from a first body end to a second body end, the body being manipulable between an open configuration and a closed configuration, the body defining:
an outer surface;
a channel extending within the body proximate the first body end and along the body axis; and
a gap extending from the outer surface into the channel, the gap being larger when the body is in the open configuration than the closed configuration the channel defining a polygon cross-section.

21. The device of claim 20, wherein the body defines a round outer surface extending along the body axis.

22. The device of claim 21, wherein the round outer surface defines a body diameter that is at least one of twice as large as a channel width defined by the channel, and at least four times as large as a channel height defined by the channel.

23. (canceled)

24. (canceled)

25. (canceled)

26. (canceled)

27. (canceled)

28. (canceled)

29. The device of claim 20, wherein the body includes a first component coupled to a second component via a hinge.

30. The device of claim 29, wherein the first component and the second component are coupled to a biasing structure configured to bias the first component towards the second component.

31. The device of claim 21, wherein the round outer surface defines a first plurality of ridges extending from the round outer surface.

32. The device of claim 20, wherein the body further defines a conical portion extending into the body proximate the second body end when the body is in the closed configuration, the conical portion being in fluid communication with the channel.

33. The device of claim 32, wherein the round outer surface tapers towards the second body end.

34. (canceled)
